(19) United States
(12) Patent Application Publication
(10) Pub. No.: US 2017/0266423 A1
(43) Pub. Date: Sep. 21, 2017

(54) MEDICAMENT CARRIER FOR THE TREATMENT AND PREVENTION OF PATHOLOGICAL CONDITIONS IN THE UROGENITAL REGION

(71) Applicant: UROBRAIN GmbH, Luzern (CH)

(72) Inventors: Jean-Jaques Becciolini, Geroldwil (CH); Otto von Stetten, Aachen (DE)

(21) Appl. No.: 15/505,085

(22) PCT Filed: Aug. 14, 2015
(86) PCT No.: PCT/DE2015/000411

§ 371 Date: Feb. 21, 2017

(30) Foreign Application Priority Data
Aug. 18, 2014 (DE) 10 2014 012 206.9

(51) Int. Cl. A61M 31/00; A61K 31/5383; A61K 9/00; A61K 31/545; A61M 25/00; A61M 25/04

(52) U.S. Cl. CPC A61M 31/002; A61M 25/0017; A61M 25/0045; A61M 25/04; A61K 9/0034; A61K 31/545; A61K 31/5383; A61M 2025/0056; A61M 2025/0213

(57) ABSTRACT

The application relates to a medicament carrier which is suitable for the treatment and prevention of pathological conditions in the urogenital region of a person, in particular in the area of the urethra. Said medicament carrier comprises a biocompatible material that cannot melt at the person's body temperature. The medicament carrier is prepared, e.g. at least partially coated, with an active substance or with a mixture of active substances. The medicament carrier is provided with a recovery string (6) for removing same from the body orifice after completion of the treatment. Active substances can be administered generally in the urogenital region using the medicament carrier. The medicament carrier can also be used for microbial reconstitution of the urogenital tract and for sterile catheterization.

The invention relates to a medicament carrier which is suitable for the treatment and prevention of pathological conditions in the urogenital region and is inserted into a body orifice of a person in the urogenital region, in particular into the urethra, i.e. the ureter. The medicament carrier can also be inserted into the vagina or even the anus.

Furthermore, with the invention is possible a method for the microbial reconstitution of the urogenital region, a method for the local administration of active substances in the urogenital region, in addition to a method for sterile catheterization, in particular of the bladder, as well as a method for anchoring a catheter in the body.

Pathological conditions of the urogenital tract, e.g. bacterial infections, nycturia, i.e. increased nocturnal urination, etc. are usually treated with systemically administered medicines, which are administered either orally or through the skin, i.e. transdermally, or through infusions of medication solutions into the veins of a patient. In most of the cases, it is thereby necessary to employ relatively high dosages of a medicine in order to achieve the desired effect. This is associated with a consequently high rate of undesired side effects.

This could be widely avoided with a local administration of the medicines

For this purpose, suppositories were proposed, which are inserted into the relatively short urethra of a female person and there release an active substance of a medicine.

In the US Patent Specification U.S. Pat. No. 5,085,650, a suppository is described, which has a cylindrical body that is shaped at one end, the proximal one, as a conical stop that, when the suppository is inserted, abuts on the entrance to the urethra and prevents the suppository from moving into the bladder. At the other, distal end a spherical thickening is provided, which, when the suppository is inserted, lies in the bladder and prevents therewith the repulsion of the suppository from the urethra. The suppository comprises a medicine which is embedded into a base material that melts slowly in the urethra and releases thereby the active substance.

The spherical thickening of the suppository can lead to irritations in the bladder, e.g. pain and an additional urge to urinate.

A suppository according to US Patent Specification U.S. Pat. No. 6,464,670 avoids this. The suppository has a stop at the proximal end, which abuts on the entrance to the urethra and prevents the suppository from moving towards the bladder. On the stop is a club-shaped body conically attached, which expands conically towards the bladder, and which, however, does not come into contact with the enervated entrance to the bladder. The conical layout prevents an expulsion of the suppository from the urethra, since appropriate muscular holding forces are exerted onto the club-shaped body.

The suppositories mentioned are made of a material, e.g. a fat, wax or oil, which melts at body temperature. Their form is not very stable and they can break in the urethra when they are being placed or during the melting operation. The fragments can reach the bladder or be expelled.

According to US Patent Specification U.S. Pat. No. 7,267,670, this is avoided by making that the suppository contains a plastic core which is joined with a widened plastic base at the proximal end, which abuts on the entrance to the urethra when the suppository is inserted. The plastic core of the suppository is removed after the melting of the applied medicine. The manipulation is laborious and complicated, since the plastic base cannot be easily grasped.

But also with this design, fragments may appear during the melting operation of the medicine, which can reach the bladder.

The proposed suppositories are only provided for insertion into the female urethra; for the male urethra, no suppository for the treatment and prophylaxis of urinary tract infections is known.

The invention is concerned with the object of specifying a medicament carrier for the treatment and/or prophylaxis of pathological conditions in the urogenital region, which remains stable during insertion in the urogenital region and also during its residence time, so that a liberation of an active substance is possible without complications.

This object, according to the invention, is solved through the features of claim 1.

According to the invention, a medicament carrier comprises a material which cannot melt at the body temperature of the treated person and is essentially rod- or club-shaped. The form of said medicament carrier is similar to the one of a suppository, however, the material of the medicament carrier cannot melt at the body temperature of the treated person. Preferably, a stop is provided at its proximal end, which abuts on the body orifice. The medicament carrier is prepared, e.g. coated, with an active substance or a mixture of active substances that cannot melt at body temperature.

The medicament carrier is shaped such that it can be inserted, e.g. into the urethra, the vagina or even into the anus, depending on the desired application.

The medicament carrier has preferably a proximal base which abuts on the entrance to the body orifice, in particular to the urethra, when the medicament carrier is inserted and prevents the medicament carrier from moving into the body orifice. On the basis is attached an active substance carrier in the distal direction, which is configured as being rod- or club-shaped and does not reach in particular the person's bladder when the urethra is being treated.

The base and the active substance carrier comprises a biocompatible material that cannot melt at the person's body temperature. The active substance carrier is prepared, e.g. at least partially coated, with an active substance or with a mixture of active substances.

Through this configuration, the medicament carrier can be easily inserted into a body orifice, e.g. the urethra, namely both of a female and a male person, and remains at the placed location.

The medicament carrier can be removed after placement and completion of the treatment, e.g. through a recovery string.

The medicament carrier for the treatment of pathological conditions in the urogenital region of a person, and in this case in particular in the area of the urethra, is configured such that the entrance to the person's bladder is not touched and no unpleasant reactions, such as urge to urinate, etc. are provoked.

According to a preferred embodiment of the invention, the active substance carrier is at least partially coated with the active substance or the mixture of active substances, or a material into which the active substance or the mixture of active substances are integrated. This coating can, without even melting, disintegrate successively or be liberated, e.g. in microflakes, during treatment. The active substance or the mixture of active substances can also be integrated into the material of the active substance carrier that cannot melt at the body temperature of the person being treated.

It is possible that the coating does not disintegrate or is not liberated, and the active substance or the mixture of active substances takes effect directly through the contact with the region being treated.

According to a further embodiment of the invention, the medicament carrier or the active substance carrier comprises a material which disintegrates or decomposes slowly during the treatment, wherein this material does not directly melt at body temperature. Suitable materials are polyactates, anhydrides, polyester, etc.

In particular in the case of a medicament carrier, preferably in the form of a suppository for a male person, attention must be paid to the fact that the suppository and, in particular, the active substance carrier is flexible within certain limits in order not to affect too strongly the movements of the urethra; on the other hand, said medicament carrier must be sufficiently rigid so that it can be inserted into the ureter.

According to a further embodiment of the invention, the active substance carrier has a through axial channel running in the longitudinal direction. As a conventional urinary catheter, this channel serves, on the one hand, to ensure drainage of urine in the case of a prolonged residence time of the medicament carrier in the urethra.

According to the invention, the channel is dimensioned such that a catheter can be pushed through the channel. For this purpose, the channel has dimensions of between 0.3 and 0.6 centimeters, in particular 0.35 to 0.45 centimeters.

Likewise, this channel is prepared, e.g. coated, with the active substance or the mixture of active substances in order not to push infectious germs into the bladder when inserting the catheter. Hereby a sterile catheterization is achieved.

Furthermore, locally acting medicines can be introduced into the body orifice, e.g. the bladder, by means of the catheter. With an appropriate configuration of the catheter, the progress of the treatment can also be observed.

Additionally, the catheter can be fixed to the medicament carrier or in the channel, so that further auxiliary means, such as a balloon, with the known irritations are not necessary for fixing the position of the catheter.

According to an embodiment of the invention, the active substance carrier is made of a mixture of medicaments that cannot melt at body temperature, in which a mucoadhesive agent is additionally incorporated, so that the active substance carrier adheres to the previously determined site in the urogenital region, in particular in the urethra, and remains there until the complete liberation of the active substance or the mixture of active substances.

The mixture of active substances used can also contain antimicrobially effective substances in order to enable a microbial reconstitution of the urogenital region, in particular the urethra. For this purpose, the medicament carrier remains for a prolonged time at the treatment site, causing undesired germs or microbes to be killed. The antimicrobial substances are released or remain adhered to the medicament carrier. Suitable substances are also, besides the usual antiseptic substances, as are usual in the industry of disinfecting agents, mixtures of heavy metals, as are commercially available, for example, from the company Bactigard. In the last case, the active substances remain on the medicament carrier and take effect directly through contact. With a medicament carrier treated in such a way, the urogenital region, in particular the urethra, can be microbially reconstituted, so that microbes are killed during the residence time of the medicament carrier in the urogenital region.

In a particular embodiment of the invention, the medicament carrier contains antibacterial substances, in particular antibiotics, such as, e.g. cephalexin or levofloxacin, etc. for the treatment of bacterial urinary tract affections.

In other embodiment, said medicament carrier contains antifungal active substances, such as, e.g. ketoconazole or fluconazole, etc. for the treatment of fungal infections in the urogenital area.

In other embodiment, said medicament carrier contains antiparasitic active substances, such as azithromycin or doxycycline, etc. for the treatment of sexually transmitted diseases, such as, e.g. infections caused by Chlamydia trachomatis.

In other embodiment, said medicament carrier contains antiviral active substances, such as, e.g. Acyclovir, etc. for the treatment of viral infections in the urogenital region.

A medicament carrier, in particular in the urethra, where applicable, coated is removed after the end of the treatment time. The dosage of the active substances or the mixtures of active substances, in particular of those that take effect in a narrow therapeutic window, can be controlled during the residence time of the medicament carrier in the urogenital region.

The medicament carrier or a catheter pushed through the same can be fixed, for instance, through a mucoadhesive agent during the treatment time.

Suitable biocompatible materials for a medicament carrier are, e.g. polyurethane, polyethylene, polypropylene, silicone, viscose, cellulose, nylon, etc. or another natural or synthetic material which can be molded into the predetermined form of the active substance carrier. Such materials can be processed by injection moulding, casting, 3D printing, polymerization inside the mould, foaming, etc.

As materials which disintegrate slowly in the urogenital region, in particular in the urethra, are considered, by way of example, polyactates and anhydrides, polyester, etc.

Other configurations of the invention are set forth in the dependent claims.

The invention is more specifically described in exemplary embodiments by reference to the drawings.

In the drawings:

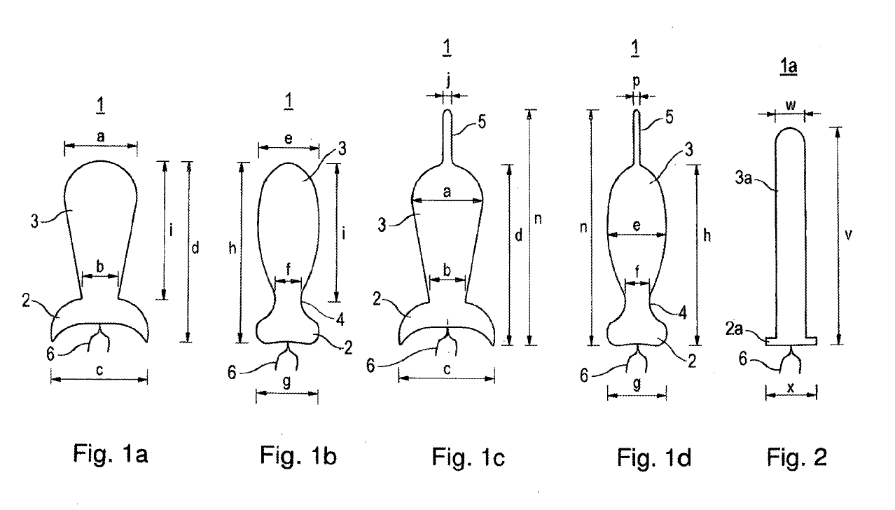

FIGS. 1A, 1B, 1C, and 1D show schematic representations, each of a medicament carrier according to the invention in the form of a suppository for being inserted into the urethra of a female person;

FIG. 2 shows a schematic representation of a medicament carrier according to the invention for being inserted into the urethra of a male person;

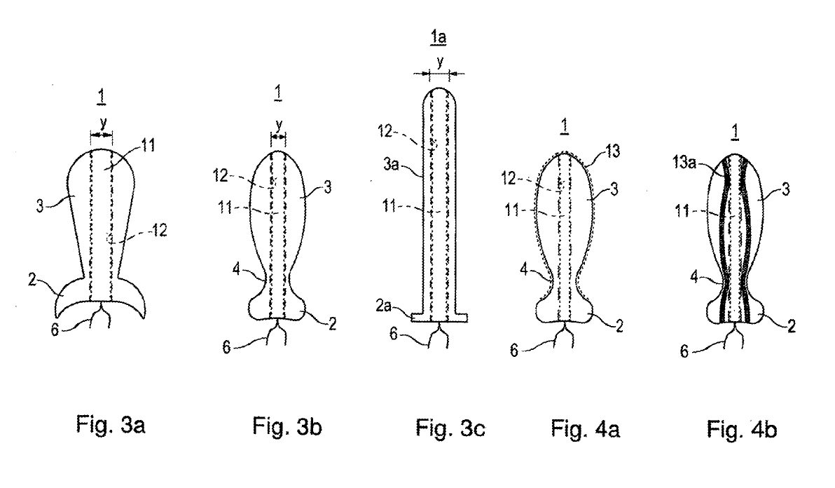

FIGS. 3A, 3B, and 3C show schematic representations of medicament carriers in the form of suppositories for being inserted into the urethra of a female person (FIG. 3A, FIG. 3B) and of a male person (FIG. 3C), respectively, each of them with a through axial channel;

FIGS. 4A and 4B show schematic representations, each of them of a medicament carrier in the form of a suppository for being inserted into the urethra of a female person, with a complete or partial coating with an active substance or a mixture of active substances;

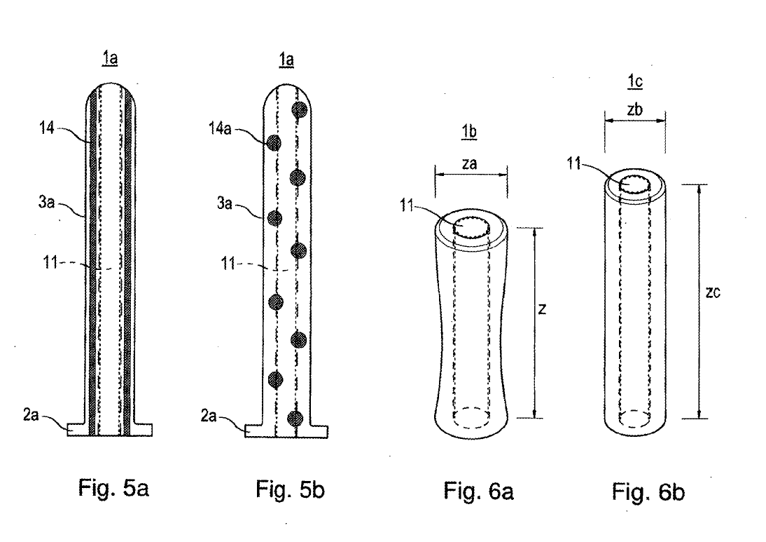

FIGS. 5A and 5B show schematic representations, each of them of a medicament carrier for being inserted into the urethra of a male person, with an axial through channel coated completely with an active substance or a mixture of active substances (FIG. 5A) or with a partial coating (FIG. 5B);

FIGS. 6A and 6B show schematic representations, each of them of a medicament carrier in the form of a tampon for being inserted into the urethra, wherein the tampon is designed to remain longer inside the urethra.

In each of FIGS. 1A, 1B, 1C and 1D, a medicament carrier 1 is represented in the form of a suppository, which is inserted into the urethra of a female person. The medicament carrier 1 has a base 2 at its proximal end which abuts on the entrance to the urethra, not shown, and covers it when the medicament carrier is inserted. An essentially club-shaped active substance carrier 3 attaches to the base 2 in the distal direction.

According to FIG. 1A, the club-shaped active substance carrier 3 expands conically from the base 2 in the distal direction and is rounded at its distal end. The total length d of the medicament carrier is designed such that the same does not reach the bladder of the treated person and ends before the entrance to the bladder in order to avoid irritations.

The medicament carrier 1 according to FIG. 1A has a length d of 3.5 to 4.5 centimeters (cm), preferably 4.0 to 4.5 cm, and has a diameter a at the distal end of 0.8 to 1.5 cm, preferably 0.8 to 1.2 cm. At the proximal side, on the base 2, the active substance carrier 3 has a diameter b of 0.4 to 1.0 cm, preferably 0.6 to 0.8 cm. The base 2 which serves as a stop for covering the entrance to the urethra has a diameter c of between 1.0 and 2.0, preferably 1.3 to 1.7 cm.

The indicated measurements may vary depending on the body structure of the person being treated. The reference numbers for the individual measurements apply to all figures.

FIG. 1B shows a medicament carrier 1 which has a form similar to a game pawn. The medicament carrier 1 again has a base 2 and an active substance carrier 3 which extends in the distal direction. Starting from the base 2, it has an area 4 firstly tapering, which expands and changes continuously into the club-shaped active substance carrier 3. The width g of the base 2 is of 0.8 to 1.5 cm, preferably of 0.8 to 1.2 cm; the area 4 has on its narrowest area a diameter f of approximately 0.4 to 1.0 cm, preferably 0.6 to 0.8 cm. The largest width e of the medicament carrier 1 in the upper distal area of the active substance carrier 3 is similar to the width g of the base 2, i.e. 0.8 to 1.5 cm. The total length h of the medicament carrier 1 is of approximately 4.0 to 4.5 cm; from the distal tip to the taper there are approximately 2.5 to 3.5 cm, preferably 2.8 to 3.2 cm (Length i).

FIG. 1C shows a medicament carrier 1 similar to the one of FIG. 1A. However, a thin axial tip 5 is provided at the distal end, which protrudes into the bladder and is also prepared with an active substance or a mixture of active substances. With this configuration of the medicament carrier, pathological conditions of the bladder can also be locally treated without the appearance of irritations using the prepared tip.

The diameter j of the tip 5 is of approximately 0.1 to 0.3 cm, preferably 0.15 to 0.25 cm, its length of approximately 1.5 to 2.5 cm, and the total length n of the medicament carrier with the tip 5 is of approximately 5.5 to 6.5 cm, preferably 5.0 to 5.5 cm.

FIG. 1D shows a medicament carrier 1 which is configured in a similar way as in FIG. 1B, i.e. with a base 2 with a diameter g of 1.0 to 2.0 cm to which a taper 4 and a club-shaped active substance carrier 3 with a maximal diameter e of 0.8 to 1.5 cm is attached. The medicament carrier 1 is, similar as in FIG. 10, provided with a tip 5 of approximately 1 to 2 cm and has a total length n of approximately 5.5 to 6.5 cm.

FIG. 2 is a medicament carrier la for being inserted into a male urethra. It has a circular base 2a which serves as a stop and abuts on the entrance to the urethra when the suppository is inserted. The active substance carrier 3a is cylindrical. The total length v of the medicament carrier is of 5.0 to 7.0 cm, preferably 5.5 to 6.6 cm; the diameter w of the active substance carrier 3a is of 0.4 to 1.0 cm, preferably of 0.6 to 0.8 cm. The diameter x of the base 2a is of 1.2 to 2.0 cm, preferably of 1.3 to 1.7 cm.

For simplifying the removal of the medicament carrier from the urethra after the treatment time, a recovery string 6 is joined in each case with the medicament carrier. This is indicated in all representations in the Figures.

FIGS. 3A, 3B, as well as 4A and 4B show medicament carriers 1 similar to those of FIGS. 1A or 1B, and FIG. 3C a medicament carrier la similar to that of FIG. 2.

Each one of the medicament carriers are added around a central, axial through channel 11. This channel 11 serves i.a. to enable urine drainage, also in the cases when the medicament carrier remains for a prolonged time in the urethra. Furthermore, a catheter can be pushed through this channel 11 when it has the appropriate dimensions, which reaches the bladder in order to, where applicable, administer medicines locally or monitor the treatment operation through an optical sensor.

In order to avoid that together with the catheter microbes are pushed into the bladder, which can cause inflammations of the bladder or even inflammations of the renal pelvis, the channel 11 is provided in each case with an antimicrobial coating 12. In this way, no microbes can penetrate into the bladder or the kidneys in the catheterization and also during the residence time of the catheter.

The diameter y of the channel 11 is of approximately 0.3 to 0.6 cm, in particular of 0.35 to 0.45 cm.

FIGS. 4A and 4B show medicament carriers 1 which are each inserted into the female urethra, wherein the active substance carrier 3 of FIG. 4A is continuously provided with a coating 13 of the active substance or the mixture of active substances. This coating 13 is very thin and can disintegrate slowly during the residence time of the medicament carrier in the urethra.

It is also possible to configure this coating 13 as a contact medicine, so that the coating 13 remains in the urethra during the whole residence time and the active substance takes effect directly through the contact with the tissue of the urethra.

This applies to all embodiments shown, also in the case where this is not represented.

FIG. 4B shows a medicament carrier 1 that, instead of a complete coating of the active substance carrier 3, has a coating 13a only on individual points, in this case in stripes.

FIGS. 5A and 5B each show a medicament carrier la for being inserted into the urethra of a male person, wherein the medicament carrier la is essentially assembled as in FIG. 2, i.e. having a circular base 2a and a cylindrical active substance carrier 3a as well as a through channel 11.

In order to better retain the medicament carrier in the female or male urethra, the medicament carriers can be coated with mucoadhesive substances, wherein the coating is applied, e.g. in stripes 14 according to FIG. 5A or in points 14a, as is shown in FIG. 5b.

Such materials are, e.g. carbomers, poloxamers, polyacrilates and derivatives, chitosans and chitosan derivatives, alginate-polyethylenglycol acrylates, thiomers (e.g. polycarbophilcysteines, etc.), lectins, as well as mixtures of the mentioned materials. Thus, the amount of mucoadhesive agent is selected such that the medicament carrier can be painlessly removed after the desired residence time.

FIGS. 6A and 6B represent the medicament carriers 1b and 1c, respectively, which are similar in their form to a commercial tampon with a low diameter. In this case, the medicament carriers are made of a mixture of medicaments which cannot melt at body temperature, in which a mucoadhesive agent is additionally incorporated or which is additionally coated with such one, so that the medicament carriers remain till the end of the treatment at the previously determined site in the urogenital region, in particular in the urethra. The medicament carrier 1b according to FIG. 6A has a length z of, e.g. 2.5 to 3.5 cm, and a diameter za of 0.8 to 1.5 cm. This medicament carrier 1b again can have a through channel 11. The medicament carrier 1c according to FIG. 6B has a length zc of approximately 5.0 to 7.0 cm and a diameter zb of 0.4 to 1.0 cm. Also in this embodiment, a through channel 11 can be provided in each case for the purposes mentioned above.

All described medicament carriers can be placed manually or with an applicator.

Methods for coating the medicament carrier with a continuous covering, with a partial covering, e.g. in stripes or on individual points, are known in the pharmaceutical science, e.g. immersion, fusing, spraying, lyophilization, etc. Where applicable, the medicament carriers are pretreated with plasma in order to achieve a better adherence. The respective active substances and dosages are apparent from the corresponding therapies.

The medicament carrier prepared or coated with an active substance is inserted into the urogenital region and is removed after a predetermined time with the recovery string 6. Since the release kinetics of the respective mixtures of medicaments is known or has been previously determined, the dose of the mixtures of medicaments can be exactly adjusted during the residence time of the medicament carrier in the urogenital region. In this way, is also achieved to accurately dose active substances with a narrow therapeutic window, in which there is a short gap between an effective and a toxic mixture of medicaments.

It is clear from the above that a prepared medicament carrier according to the invention can be used in general for the administration of an active substance or a mixture of active substances or others, such as antimicrobial substances, in order to treat pathological conditions of the urogenital region or reconstitute the same.

1. A medicament carrier which is suitable for the treatment and prophylaxis of pathological conditions in a urogenital region of a person comprising: a biocompatible material which cannot melt at the person's body temperature, wherein the medicament carrier is prepared with an active substance or a mixture of active substances for the treatment of the urogenital region.

2. The medicament carrier according to claim 1, wherein the active substance or the mixture of active substances cannot melt at the person's body temperature.

3. The medicament carrier according to claim 1, wherein the medicament carrier is at least partially coated with the active substance or the mixture of active substances.

4. The medicament carrier according to claim 1, wherein the medicament carrier is rod- or club-shaped for being inserted into a body orifice in the urogenital region.

5. The medicament carrier according to claim 4, wherein the medicament carrier comprises (i) a proximal base which serves as a stop for covering the body orifice and (ii) a distal rod- or club-shaped active substance carrier, which is prepared with the active substance or the mixture of active substances, and is adapted for being inserted into the body orifice.

6. The medicament carrier according to claim 5, wherein the active substance or the mixture of active substances is integrated in material of the active substance carrier.

7. The medicament carrier according to claim 1, wherein the active substance or the mixture of active substances is present in a material which slowly disintegrates at the person's body temperature.

8. The medicament carrier according to claim 1, wherein the medicament carrier has an through channel.

9. The medicament carrier according to claim 8, wherein the channel is dimensioned such that a catheter can be pushed through it.

10. The medicament carrier according to claim 9, adapted to allow fixation of the catheter can be fixed in the channel.

11. The medicament carrier according to claim 8, wherein the channel is prepared with the active substance or the mixture of active substances and/or an antimicrobial substance.

12. The medicament carrier according to claim 1, wherein the medicament carrier has an axial tip at a distal end, which is prepared with the active substance or the mixture of active substances.

13. The medicament carrier according to claim 1, wherein the medicament carrier has a tapering area attached to a base, which is adapted to expands and changes into a rod- or club-shaped contour.

14. The medicament carrier according to claim 1, wherein the medicament carrier is essentially completely, in stripes, or at individual points coated with the active substance or the mixture of active substances.

15. The medicament carrier according to claim 14, wherein, said medicament carrier is provided with mucoadhesive substances adapted to fix the medicament carrier in the body orifice, which substances are, where applicable, integrated in the active substance or the mixture of active substances.

16. The medicament carrier according to claim 15, wherein the amount of mucoadhesive substances is selected such that the medicament carrier can be painlessly removed after a predetermined time.

17. The medicament carrier according to claim 14, wherein the medicament carrier is prepared with antimicrobial substances which are released or stayfixedly adhered to the medicament carrier during a residence time of the medicament carrier in the body orifice.

18. The medicament carrier according to claim 17, wherein the medicament carrier is adapted to be inserted into an urethra of the person and to remain there for a period of time that kills undesired germs and guards against infections.

19. The medicament carrier according to claim 1, wherein said medicament carrier comprises a biocompatible base material which slowly disintegrates in the urogenital region.

20. The medicament carrier according to claim 1, wherein a dosage of the active substance or mixture of active substances can be controlled via a residence time of the medicament carrier in the body orifice.

21. The medicament carrier according to claim 1, wherein said medicament carrier contains antibacterial substances for the treatment of bacterial urinary tract diseases.

22. The medicament carrier according to claim 1, wherein said medicament carrier contains:
antifungal active substances treating fungal infections in an urogenital area, antiparasitic active substances for treating sexually transmitted diseases including infections caused by Chlamydia trachomatis, or antiviral active substances for treating viral infections in the urogenital region.

23. The medicament carrier according to claim 22, wherein said antifungal active substances are ketoconazole or fluconazole, said antiparasitic active substances are azithromycin or doxycycline and said antiviral active substance includes acyclovir.

24. The medicament carrier according to claim 1, wherein urogenital region is an urethra area.

25. The medicament carrier according to claim 1, wherein the medicament carrier is equipped with a recovery string for removing it from an body orifice.

26. A method for administering an active substance or a mixture of active substances to a person in need thereof comprising:
providing the medicament carrier of claim 1 comprising, the active substance or the mixture of active substances and inserting the medicament carrier into the urogenital region of the person and removing it after a predetermined time.

27. The method of claim 26, wherein the person is in need of microbial reconstitution of the urethra, the medicament carrier is prepared with antimicrobial substances, is inserted into the urethra, and remains there for a period of time sufficient to kill undesired germs and guard against infections.

28. The method of claim 26, wherein the medicament carrier has a through canal adapted to receive a catheter and is prepared with antimicrobial substances, wherein the medicament carrier is administered to achieve sterile catheterization.

29. The medicament carrier according to claim 19, wherein the biocompatible base material is a polyactate, anhydride or polyester material.

30. The medicament carrier according to claim 21, wherein the antibacterial substances are antibiotics including cephalexin or levofloxacin.
